(19) United States
(12) Patent Application Publication
(10) Pub. No.: US 2015/0342897 A1
(43) Pub. Date: Dec. 3, 2015

(54) POLYPEPTIDE LOADED POCA NANOPARTICLES FOR ORAL ADMINISTRATION

(71) Applicant: Glaxo Group Limited, Brentford, Middlesex (GB)

(72) Inventors: Sean Matthew Cleveland, Stevenage (GB); Mark Andrew Paulik, Research Triangle Park, NC (US); Stefan Salomon, Stevenage (GB)

(73) Assignee: Glaxo Group Limited, Brentford, Middlesex (GB)

(21) Appl. No.: 14/401,333

(22) PCT Filed: May 16, 2013
(86) PCT No.: PCT/IB2013/000964

§ 371 Date: Nov. 14, 2014

Related U.S. Application Data
(60) Provisional application No. 61/647,633, filed on May 16, 2012.

(51) Int. Cl. A61K 9/51; A61K 38/22; A61K 9/00

(52) U.S. Cl. CPC A61K 9/5138; A61K 9/0053; A61K 38/22; A61K 9/5192

(57) ABSTRACT

Nanoparticles comprising biologically active peptides and poly(octylcyanoacrylate) as well as related subject matter is disclosed.

### BACKGROUND OF THE DISCLOSURE

The vast majority of biopharmaceuticals, particularly protein and peptide therapeutics, are administered by the parenteral route, e.g. by intravenous or subcutaneous injection. These routes of administration can often be inconvenient and painful which reduces patient compliance, particularly when multiple injections per day are required. They can also be costly (e.g. administration via intravenous infusion requires visits to a medical centre).

Oral administration of biopharmaceuticals would overcome many of these drawbacks but has its own challenges. Such molecules are not normally orally bioavailable since proteins and peptides are subject to proteolytic degradation in the protease rich environment of the stomach and intestine. Other obstacles include: the stability of the molecule in the acidic conditions encountered in regions of the gastrointestinal (GI) tract; the time delay between the drug entering the GI tract and reaching its target and, in the case of a protein or peptide therapeutic which requires systemic exposure, permeability of the drug across biological membranes, such as the intestinal mucosa (Goldberg and Gomez-Orellana, Nat Rev Drug Discov (2003) 2(4): 289-295). For these reasons, there are currently no orally delivered biopharmaceuticals on the market with the exception of the unusual 11 amino acid cyclic peptide cyclosporin. Cyclosporin is not a representative peptide in that it is water insoluble, a cyclic peptide, a nonribosomal peptide, and contains a single D-amino acid (rarely encountered in nature).

To protect the active protein or peptide and allow its absorption through the gut wall a sophisticated drug delivery method is required to bypass metabolic processes and preserve the intact and functional biopharm (Hamman J. H., Enslin G. M., Kotz A. F. (2005), Oral Delivery of Peptide Drugs, Biodrugs 165-177).

Particulate carriers such as microspheres, liposomes, and various nanoparticles are being explored for oral delivery of biopharmaceuticals. However, such technologies may not be suitable for oral delivery due to their size, instability within the stomach/GI tract and low drug loading.

Metabolic diseases and disorders, such as obesity are on the increase. Obesity is an emerging global problem affecting populations not only of developed countries but is now becoming more and more prevalent in low- and middle-income countries. The global expenditure for the treatment of obesity and obesity-related diseases and the provision of diet nutrients is constantly increasing (World Health Organisation (2011), Obesity and overweight, Factsheet no. 311 (updated March 2011); James P. T., Leach R., Kalamara E., Shayeghi M. (2001), Worldwide Obesity Epidemic, Obesity Research 9(4): 228S-233S). The treatment options for obesity are limited and are mainly restricted to surgical interventions such that there is a clear clinical need to identify viable drug based treatment options. Exendin-4, a non-human incretin mimetic with greater potency than native GLP-1, is currently licensed (BYETTA™, BYDUREON™) for the treatment of type 2 diabetes and is also under investigation in numerous clinical trials for the treatment of obesity. However, almost all late stage metabolic peptides known to be in development, such as Exendin-4, require injections.

### SUMMARY OF THE DISCLOSURE

A nanoparticle comprising a biologically active polypeptide, wherein the nanoparticle comprises poly(octylcyanoacrylate), otherwise known as POCA, is provided, in particular for oral administration.

A population of nanoparticles comprising nanoparticles of the disclosure, wherein at least 90% of nanoparticles by number have a hydrodynamic diameter within 10 nm to 200 nm as measured by dynamic light scattering techniques, is also provided.

The disclosure further provides a method of producing nanoparticles comprising the steps of: a) dissolving octylcyanoacrylate (OCA) in an organic solvent to form a monomer solution;

b) adding the monomer solution from step (a) to an acidic aqueous solution to form an emulsion of organic droplets in an aqueous phase; and simultaneously or sequentially

c) adding an aqueous solution of a biologically active polypeptide to the emulsion from step (b) and allowing polymerisation of the monomer; and

d) allowing the organic phase to evaporate, thereby obtaining an aqueous suspension of poly(octylacrylate) (POCA) nanoparticles containing the polypeptide.

A pharmaceutical composition comprising nanoparticles of the disclosure is also provided.

A use of nanoparticles of the disclosure comprising a metabolic peptide for treating any one or more of the following metabolic diseases is further provided: a disorder associated with elevated glucose levels, diabetes (type 1 or 2 or gestational), metabolic syndrome, hyperglycemia, impaired glucose tolerance, beta cell deficiency, and a disease characterised by or associated with overeating, such as obesity.

Also provided is a method of treating a subject having one or more of the following metabolic diseases: a disorder associated with elevated glucose levels, diabetes (type 1 or 2 or gestational), metabolic syndrome, hyperglycemia, impaired glucose tolerance, beta cell deficiency, and a disease characterised by or associated with overeating, such as obesity, by administering a therapeutically effective amount of nanoparticles according to the disclosure.

### BRIEF DESCRIPTION OF THE FIGURES

FIG. 1 is a bar chart showing the stability of exendin-4 POCA nanoparticles and free exendin-4 at various time points in simulated gastric fluid.

FIG. 2 is a bar chart showing the stability of exendin-4 POCA nanoparticles and free exendin-4 at various time points in simulated intestinal fluid.

FIG. 3 shows the percentage change in blood glucose levels at various time points (prior to and 0.5, 1, 2, 3, 4 and 8 hours post dose) in C57BL/6 mice intravenously administered with either exendin-4 POCA nanoparticles, free exendin-4 or a saline control.

FIG. 4 shows the percentage reduction in food intake relative to water control at 12, 24 and 36 hour time points post dose in C57/BL6 fed mice administered orally with either exendin-4 POCA nanoparticles or free exendin-4, or administered subcutaneously with free exendin-4.

FIG. 5 relates to a dose-range study and shows the percentage reduction in food intake relative to water control at 12, 24 and 36 hour time points post dose in C57/BL6 fed mice administered orally with either 20 mg/kg, 10 mg/kg, 5 mg/kg or 2.5 mg/kg exendin-4 POCA nanoparticles or 20 mg/kg free exendin-4.

FIG. 6 relates to a dose-range study and shows the percentage reduction in body weight relative to water control at 12, 24 and 36 hour time points post dose in C57/BL6 fed mice administered orally with either 20 mg/kg, 10 mg/kg, 5 mg/kg or 2.5 mg/kg exendin-4 POCA nanoparticles or 20 mg/kg free exendin-4.

### DETAILED DESCRIPTION

There is a strong need for an efficient and effective means of orally administering biopharmaceuticals. In particular, it would be desirable to find a means of orally administering biopharmaceuticals which achieves desirable release kinetics and systemic bioavailability, whilst maintaining protein/peptide stability and activity.

The present disclosure provides a solution to these problems. The present disclosure provides nanoparticles comprising poly(octylcyanoacrylate) for oral administration of a biologically active polypeptide, in particular a metabolic peptide, such as exendin-4. We have orally administered exendin-4 peptide using the nanoparticles of the disclosure and achieved significant biological effects including lowered blood glucose levels, reduced food intake and weight loss indicating the therapeutic potential of the metabolic peptide loaded nanoparticles of the disclosure. Accordingly, nanoparticles of the disclosure can achieve systemic delivery of biologically active polypeptides administered by the oral route. Nanoparticles of the disclosure may also achieve topical delivery of biologically active polypeptides to areas of the gastrointestinal (GI) tract.

In an embodiment, a nanoparticle has a hydrodynamic diameter of 300 nm or less. In another embodiment, a population of nanoparticles is provided wherein at least 90% of nanoparticles by number have a hydrodynamic diameter within 10 nm to 200 nm as measured by dynamic light scattering techniques.

A nanoparticle formulation of the disclosure may also further comprise oligofructose (OFS).

The present disclosure also encompasses methods of producing such nanoparticles, pharmaceutical compositions comprising such nanoparticles and methods of treating metabolic disorders, such as obesity, using such nanoparticles.

The disclosure provides a method of producing nanoparticles comprising the steps of:

a) dissolving octylcyanoacrylate (OCA) in an organic solvent to form a monomer solution;

b) adding the monomer solution from step (a) to an acidic aqueous solution to form an emulsion of organic droplets in an aqueous phase; and simultaneously or sequentially

c) adding an aqueous solution of a biologically active polypeptide to the emulsion from step (b) and allowing polymerisation of the monomer; and

d) allowing the organic phase to evaporate, thereby obtaining an aqueous suspension of poly(octylacrylate) (POCA) nanoparticles containing the polypeptide.

In an embodiment, the organic solvent is selected from the group consisting of: ethylacetate, dichloromethane, and chloroform. Other water immiscible solvents may also be used.

In an embodiment the acid used to generate the acidic aqueous solution used in step (b) is selected from the group consisting of: hydrochloric acid, sulphuric acid, nitric acid and citric acid. In a particular embodiment the acid used is hydrochloric acid.

In an embodiment, the pH of the aqueous solution used in step (b) is in the range of 1-3.5, or 2-3.5. In an embodiment, the pH used is about 2.

In an embodiment, the aqueous solution of step (b) comprises a surfactant and/or a stabiliser. The surfactant may be any one selected from the group consisting of: a poloxomer, a polysorbate (e.g. TWEEN™) surfactant, a macrogol ether (e.g. BRIJ™) surfactant, polyvinyl alcohol (PVA), and polyvinylpyrrolidone (PVP). The stabiliser may be any one selected from the group consisting of: dextran, chitosan, fucoidan, pectin, glycogen, amylase, and amylopectin.

In an embodiment, a step to neutralise the emulsion is included between steps (c) and (d). The emulsion may be neutralised using sodium hydroxide.

The ratio of polypeptide to monomer may be in the range of 0.1 to 25% w/w. In an embodiment the ratio of polypeptide to monomer is 1-10% w/w.

Allowing the organic phase to evaporate may be passive or active. For example active evaporation may be by the use of heat.

Alternatively, known technologies, such as Liquidia Tecnologies PRINT™ technology (as described in U.S. Pat. No. 7,976,759 and WO2007024323), may be used to formulate nanoparticles of the disclosure.

A pharmaceutical composition of the disclosure may further comprise oligofructose (OFS).

“Nanoparticles” as used herein are submicron sized particles such as for example 1-1000 nm. Naonparticles having a diameter of less than 200 nm are particularly suitable for oral administration for systemic exposure. In an embodiment nanoparticles for oral administration for systemic exposure are 5-100 nm in diameter.

“Systemic exposure” as used herein is intended to mean the delivery of the biologically active agent, e.g. peptide or protein, to the systemic system (e.g., bloodstream) by absorption through the GI tract epithelium and/or Peyer's patches.

Polyalkyl cyanoacrylate (PACA) nanoparticles are biocompatible, biodegradable, and stable within simulated gastric and intestinal fluids. PACA nanoparticles can also be used to modulate the release profile of the encapsulated molecule, using different length alkyls chain and size of particulates, and different conditions and methods of preparation. We have shown that biologically active polypeptide loaded poly(octylcyanoacrylate) (POCA) nanoparticles provide a desirable systemic pharmacological response when administered orally, whilst maintaining polypeptide stability and activity.

“Oral administration” as used herein refers to the administration of nanoparticles, and compositions of the disclosure by mouth. Nanoparticles and compositions of the disclosure are typically swallowed and travel through the gastrointestinal (GI) tract where they are absorbed across the intestinal mucosa into the circulation for systemic action. Absorption may begin in the mouth (buccal cavity) and stomach, but usually occurs in the small intestine.

The “gastrointestinal (GI) tract” includes the upper GI tract: mouth, pharynx, oesophagus and stomach; and the lower GI tract: small intestine, duodenum, jejunum, ileum, large intestine (cecum, colon—including the ascending colon, transverse colon, descending colon and sigmoid flexure), rectum and anus; as well as the gall bladder, liver and pancreas. Nanoparticles of the disclosure may target any one or more of the aforementioned regions of the GI tract.

The term “biologically active agent” as used herein is a term used to indicate that the molecule must be capable of at least some biological activity when reaching the desired target. For the avoidance of doubt, the term “biologically active agent” and the term “biologically active molecule” as used throughout the specification are intended as to have the same meaning and are used interchangeably. Biologically active agents include proteins, peptides, and oligonucleotides. “Oligonucleotides” include mRNA, antisense RNA and DNA, siRNA, miRNA agonists and antagonists, and RNA and DNA aptamers. In a particular embodiment the biologically active agent of the disclosure is a “biologically active polypeptide”, which encompasses both a “biologically active protein” and a “biologically active peptide”. In an embodiment, the biologically active polypeptide is a polypeptide of 20 kDa or less in size, in particular 18 kDa or less, 15 kDa or less, 12 kDa or less, or 10 kDa or less. In an embodiment, the biologically active polypeptide comprises 70 or fewer amino acid residues. In a specific embodiment, the biologically active polypeptide comprises or consists of a metabolic peptide.

A “polypeptide” in its broadest sense is a polymer of amino acids joined together by peptide bonds. A polypeptide includes both proteins and peptides.

The term “protein” as used throughout this specification includes polypeptides having a molecular weight of at least 11 kDa, or at least 12 kDa, or at least 50 kDa, or at least 100 kDa, or at least 150 kDa or at least 200 kDa. Proteins for encapsulation may also be of considerable length such as at least 70 amino acids in length or at least 100 amino acids in length or at least 150 amino acids in length or at least 200 amino acids in length.

The term “peptide” as used throughout this specification refers to a molecule comprising two or more amino acid residues and includes shorter sequences of amino acids (compared to proteins) having a molecular weight of no more than about 10 kDa, or no more than about 8 kDa, or no more than about 5 kDa, or no more than about 2 kDa or no more than about 1 kDa or is less than 1 kDa. In an embodiment, peptides for encapsulation are no more than 70 amino acids in length or are no more than 60 amino acids in length, or are no more than are no more than 50 amino acids in length, or are no more than are no more than 40 amino acids in length, or are no more than are no more than 30 amino acids in length, or are no more than 20 amino acids in length or are less than 10 amino acids in length.

A “metabolic peptide” as used here is any energy regulating hormone secreted from any endocrine/neuroendocrine organ. Metabolic peptides include insulinotropic peptides, incretins and various gut peptides. For example, metabolic peptides include, but not limited to, GLP-1 agonist molecules, including GLP-1 and exendin molecules, Adiponectin, Adrenomodulin, Adropin, Apelin, Amylin, Bombesin, Calcitonin and Calcitonin gene related peptide (CGRP), Cocaine- and amphetamine-regulated transcript (CART), Cholecystokinin (CCK), Des-acyl-ghrelin, Enterostatin, Endothelin, Galanin-like peptide (GALP), Gastrin-releasing peptide (GRP), Glicentin, glucagon, Glucose-dependent insulinotropic peptide (GIP), Glucagon-like peptide-2 (GLP-2), insulin, intermedin, leptin, motilin, Melanocortin agonist peptide (MTII), Neuromedin B, Neurotensin, Neuromedin U (NMU), Obestatin, Orexin A, Orexin B, oxyntomodulin (OXM), oxytocin, pituitary adenylate cyclase activating polypeptide (PACAP-38), pancreatic polypeptide (PP), PYY (PYY1-36, PYY3-36 or PYY13-36), Peptide W, secretin, stresscopin, Thyrotropin-releasing hormone (TRH), Urocortin, vasoactive intestinal peptide (VIP) and Xenin. In an embodiment, the metabolic peptide is an insulinotropic peptide or an incretin. In an embodiment, the metabolic peptide is a GLP-1 agonist, PYY, NMU, or CCK. In a particular embodiment the metabolic peptide is exendin-4.

The term “insulinotropic agent” as used herein means a compound which is able to stimulate, or cause the stimulation of, the synthesis or expression of, or the activity of the hormone insulin. Known examples of insulinotropic agents include, but are not limited to glucose, GIP, GLP-1, exendin molecules, and OXM.

The term “incretin” as used herein means a type of gastrointestinal hormone that causes an increase in the amount of insulin released when glucose levels are normal or particularly when they are elevated. By way of example they include GLP-1, GIP, OXM, PYY (e.g. PYY 3-36), VIP, and PP.

“GLP-1 agonist molecule” as used herein means any molecule capable of agonising the GLP-1 Receptor. These include but are not limited to, any polypeptide which has at least one GLP-1 activity, including GLP-1, Exendin-3, Exendin-4, oxyntomodulin, and fragments and/or variants and/or conjugates thereof, for example GLP-1(7-37), that are biologically active. In an embodiment, the GLP-1 agonist molecule is GLP-1(7-37). In an embodiment, the GLP-1 agonist molecule is GLP-1(7-37) ABG. In an embodiment the GLP-1 agonist molecule is GLP-1(3-36). In an embodiment the GLP-1 agonist molecule is GLP-1(7-36). In an embodiment, the GLP-1 agonist molecule is Syncria™ (albiglutide). In another embodiment, the GLP-1 agonist molecule is Victoza™ (liraglutide).

WO05/027978 discloses GLP-1 derivatives having a protracted profile of action. WO 02/46227 discloses heterologous fusion proteins comprising a polypeptide (for example, albumin) fused to GLP-1 or analogues and such GLP-1 analogues can be used in the present disclosure. WO05/003296, WO03/060071, WO03/059934 disclose amino fusion protein wherein GLP-1 has fused with albumin to attempt to increase the half-life of the hormone.

International Patent Application No. WO 91/11457 (Buckley et al.) discloses analogues of the active GLP-1 peptides 7-34, 7-35, 7-36, and 7-37 which can also be useful as GLP-1 agonist molecules according to the present disclosure.

An “exendin molecule” as used herein includes both exendin-3, exendin-4 and exendin related molecules.

The term “exendin-4” as used herein means exendin-4 (1-39), an exendin-4 analogue, a fragment of exendin-4 peptide, an exendin-4 derivative or a derivative of an exendin-4 analogue. The sequence of exendin-4 (1-39) is HGEGTFTSDLSKQMEEEAVRLFIEWLKNGGPSSGAPPPS (SEQ ID NO:1)

Exendin-analogues that are useful for the present disclosure are described in PCT patent publications WO 99/25728 (Beeley et al.), WO 99/25727 Beeley et al.), WO 98/05351 (Young et al.), WO 99/40788 (Young et al.), WO99/07404 (Beeley et al), and WO 99/43708 (Knudsen et al). In an embodiment the metabolic peptide is exendin-4, e.g. BYETTA™ (exenatide).

Further Exendin-analogs that are useful for the present disclosure are described in PCT patent publications WO 99/25728 (Beeley et al.), WO 99/25727 Beeley et al.), WO 98/05351 (Young et al.), WO 99/40788 (Young et al.), WO 99/07404 (Beeley et al), and WO 99/43708 (Knudsen et al).

The phrase “biologically active agent loaded nanoparticle” as used herein refers to nanoparticles in which the biologically active agent, e.g. a metabolic peptide such as exendin-4, is present either on the surface of the nanoparticle, within the nanoparticle, or both on the surface and within the nanoparticle. For example, the biologically active agent, may be incorporated into nanoparticles during the polymerisation process e.g. the biologically active agent is dissolved in the polymerisation medium, or by sorption of the biologically active agent onto and into the nanoparticles once polymerisation is complete.

Biologically active agents of the disclosure may be engineered to improve stability e.g. protease resistant proteins.

Biologically active agents of the disclosure may also be conjugated to other agents, e.g. polyethylene glycol (PEG), in order to increase half-life.

It is well recognised in the art that certain amino acid substitutions are regarded as being “conservative”. Amino acids are divided into groups based on common side-chain properties and substitutions within groups that maintain all or substantially all of the binding affinity of the antigen binding protein are regarded as conservative substitutions, see Table 1 below:

| Side chain | Members |
|---|---|
| Hydrophobic | Met, Ala, Val, Leu, Ile |
| Neutral hydrophilic | Cys, Ser, Thr |
| Acidic | Aap, Glu |
| Basic | Asn, Gln, His, Lys, Arg |
| Residues that influence | Gly, Pro |
| chain orientation | |
| Aromatic | Trp, Tyr, Phe |

The term “Light scattering techniques” as used herein is a means used to determine the size distribution profile of small particles in solution—one example of light scattering technique is dynamic light scattering which may be used to measure nanoparticles and another example of light scattering is static light scattering or low angle light scattering which may be used to measure microspheres.

The term “Dynamic light scattering” (DLS) as used herein is a method which utilises the light scattered by particle dispersions to derive information on the size of the particles. Dynamic light scattering relies on the fact that when in liquid suspension, the Brownian motion of particles is dependent on particle size and that the Brownian motion of the particles produces fluctuations in the intensity of light scattered from a particle sample. The particle diameter is derived by analysing these fluctuations by means of a correlation function. The Stokes-Einstein equation is then applied to yield the mean hydrodynamic diameter of the particles. A multi-exponential analysis can produce a size distribution, providing insight into the presence of different species inside a sample. DLS is generally accepted for the analysis of nanoparticles.

The biologically active agent encapsulated in nanoparticles and/or compositions of the present disclosure retains at least some biological activity, for example 50%, 60%, 70%, 80% or 90%, on its release from the nanoparticle e.g. into the systemic circulation. For example, when the agent is metabolic peptide a proportion of the agents in the composition retain at least some ability to bind to their target receptors/effector molecules and elicit a biological response once released from the nanoparticles. Where binding to specific target receptors/effectors is measured, such binding can be measured in a suitable biological binding assay, including but are not limited to ELISA and BIACORE™. In an embodiment the agent retains at least 50% of its affinity for the target, or at least 70% or at least 90% of its affinity (e.g. as measured by equilibrium dissociation constant KD) for the target on release from the nanoparticles when measured by a biological binding assay. The composition will be capable of eliciting a therapeutic effect in the subject to which it is administered. The biological activity of the compositions of the disclosure can be measured by any suitable assay which measures activity of the encapsulated biologically active molecule, for example where the biologically active molecule is a metabolic peptide such as exendin-4 methods for measuring a reduction in blood glucose levels, food intake and/or body weight may be used e.g. as described in Examples 3-5.

Examples of organic solvents suitable for use with the methods of the disclosure include but are not limited to water-immiscible esters such as ethyl acetate, isopropyl acetate, n-propyl acetate, isobutyl acetate, n-butyl acetate, isobutyl isobutyrate, 2-ethylhexyl acetate, ethylene glycol diacetate; water-immiscible ketones such as methyl ethyl ketone, methyl isobutyl ketone, methyl isoamyl ketone, methyl n-amyl ketone, diisobutyl ketone; water-immiscible aldehydes such as acetaldehyde, n-butyraldehyde, crotonaldehyde, 2-ethylhexaldehyde, isobutylaldehyde and propionaldehyde; water-immiscible ether esters such as ethyl 3-ethoxypropionate; water-immiscible aromatic hydrocarbons such as toluene xylene and benzene; water-immiscible halohydrocarbons such as 1,1,1 trichloroethane; water-immiscible glycol ether esters such as propylene glycol monomethyl ether acetate, ethylene glycol monoethyl ether acetate, ethylene glycol monobutyl ether acetate, diethylene glycol monobutyl ether acetate; water-immiscible phthalate plasticisers such as dibutyl phthalate, diethyl phthalate, dimethyl phthalate, dioctyl phthalate, dioctyl terephthalate, butyl octyl phthalate, butyl benzyl phthalate, alkyl benzyl phthalate; water-immiscible plasticisers such as dioctyl adipate, triethylene glycol di-2-ethylhexanoate, trioctyl trimellitate, glyceryl triacetate, glyceryl/tripropionin, 2,2,4-trimethyl-1,3-pentanediol diisobutyrate, methylene chloride, ethylacetate or dimethylsulfoxide, carbon tetrachloride, chloroform, cyclohexane, 1,2-dichloroethane, dichloromethane, diethyl ether, dimethyl formamide, heptane, hexane and other hydrocarbons, methyl-tert-butyl ether, pentane, toluene, 2,2,4-trimethylpentane, 1-octanol and its isomers or benzyl alcohol. In an embodiment, the organic solvent is selected from the group consisting of ethylacetate, dichloromethane, and chloroform. In an embodiment, the organic solvent is ethylacetate. In an embodiment, the organic solvent is a water immiscible solvent.

Examples of surfactants suitable in the present disclosure include but are not limited to: sodium cholate, poloxamer 188 (pluronic F68™, or F127), polyvinyl alcohol, polyvinyl pyrrolidone, polysorbate 80, dextrans. poloxamers, poloxamines, carboxylic acid esters of multifunctional alcohols, alkoxylated ethers, alkoxylated esters, alkoxylated mono-, di and triglycerides, alkoxylated phenols and diphenols, ethoxylated ethers, ethoxylated esters, ethoxylated triglycerides, substances of the GenapolR™ and BaukiR™ series, metal salts of fatty acids, metal salts of carboxylic acids, metal salts of alcohol sulfates, and metal salts of fatty alcohol sulfates and metal salts of sulfosuccinates and mixtures of two or more of said substances. In an embodiment, the surfactant is selected from the group consisting of a poloxomer, such as PLURONIC™ F68, a polysorbate (e.g. TWEEN™) surfactant, a macrogol ether (e.g. BRIJ™) surfactant, polyvinyl alcohol (PVA), and polyvinylpyrrolidone (PVP).

Examples of stabilisers suitable in the present disclosure include but are not limited to polysaccharides such as dextran, chitosan, fucoidan, pectin, glycogen, amylase, amylopectin.

Inulin is a non-digestible, fermentable, soluble polysaccharide fibre consisting of chains of d-fructose molecules connected by 13201 binds with a terminal al-2 linked d-glucose. Inulin chain length is highly variable and can range from 10 to 60 fructose molecules (a “Degree of Polymeristaion, or DP, of 10 to 60). Inulin is found in a wide range of plants, including Jerusalem artichokes, chicory, onions, garlic, and asparagus. Oligofructose (OFS) is inulin that has been further hydrolysed to produce a micture of medium- and short-chain molecules. In some instances, molecules that are enzymatically synthesized from smaller sugar molecules to form short or medium chains are also referred to as OFS. Fructo-oligosaccharide (FOS) is a term that generally refers to even shorter fructose-chain molecules, although it is sometimes used interchangeably with OFS.

Purified preparations of biologically active polypeptide or peptide loaded nanoparticles as described herein may be incorporated into pharmaceutical compositions for use in the treatment of the human diseases, disorders and conditions described herein. The terms diseases, disorders and conditions are used interchangeably.

The pharmaceutical preparation may comprise nanoparticles as described herein in combination with a pharmaceutically acceptable carrier. The nanoparticles may be administered alone, or as part of a pharmaceutical composition. Oligofructose (OFS) may be included in the pharmaceutical composition.

Typically such compositions comprise a pharmaceutically acceptable carrier as known and called for by acceptable pharmaceutical practice, see e.g. Remingtons Pharmaceutical Sciences, 16th edition (1980) Mack Publishing Co. Examples of such carriers include sterilised carriers such as saline or dextrose solution, optionally buffered with suitable buffers to a pH within a range of 5 to 8.

In an embodiment, pharmaceutical compositions of the disclosure are to be administered orally. A variety of dosage forms are contemplated, including liquids (solutions, suspensions (aqueous or oily), and emulsions), semi-solids (pastes), films and solids (tablets, lozenges, capsules, powders, crystals and granules). In one aspect the compositions can be administered as a drink, for example marketed as a weight loss drink for obesity treatment.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate.

The present disclosure provides a composition, in particular a pharmaceutical composition, comprising nanoparticles according to the disclosure. In an embodiment at least about 90% of the nanoparticles by number have a hydrodynamic diameter within the range of about 1 nm to about 400 nm, or about 1 nm to about 300 nm, or about 1 nm to about 280 nm, or about 1 nm to about 250 nm, or about 1 nm to about 200 nm, or about 1 nm to about 150 nm, or about 10 nm to about 300 nm, or about 10 nm to about 250 nm, or about 100 nm to about 300 nm, or about 40 nm to about 150 nm, or about 100 nm to about 300 nm, or about 100 nm to about 200 nm, or about 100 nm to about 150 nm, when measured using dynamic light scattering techniques. In a particular embodiment at least about 90% of the nanoparticles by number have a hydrodynamic diameter within the range of about 10 nm to about 300 nm. In a particular embodiment at least about 90% of the nanoparticles by number have a hydrodynamic diameter within the range of about 10 nm to about 200 nm. In a particular embodiment at least about 90% of the nanoparticles by number have a hydrodynamic diameter within the range of about 10 nm to about 150 nm.

Effective doses and treatment regimes for administering biologically active polypeptides are generally determined empirically and may be dependent on factors such as the age, weight and health status of the patient and disease or disorder to be treated. Such factors are within the purview of the attending physician.

Dosage ranges for metabolic peptides (e.g. GLP-1, Exendin-4, or PYY) may be from 0.1 mg-100 mg.

The pharmaceutical composition may comprise a kit of parts of the biologically active polypeptide loaded nanoparticles as described herein with other medicaments, optionally with instructions for use. For convenience, the kit may comprise the reagents in predetermined amounts with instructions for use.

Nanoparticles and associated pharmaceutical compositions of the disclosure may be used to treat a wide variety of diseases and conditions depending on the biologically active polypeptide loaded therein. In particular, metabolic peptides can be used to treat metabolic disorders e.g. those associated with elevated glucose levels, diabetes (type 1 or 2 or gestational), metabolic syndrome, hyperglycemia, impaired glucose tolerance, beta cell deficiency and diseases characterised by or associated with overeating, such as obesity. In a particular embodiment, exendin-4 loaded nanoparticles are used to treat obesity.

Combinations of two or more metabolic peptides may be administered in therapeutic regimens of the disclosure. In an embodiment, a population of nanoparticles comprising a first metabolic peptide may be administered in combination with a population of nanoparticles comprising a second metabolic peptide. For example, a population of nanoparticles comprising exendin-4 may be administered in combination with a population of nanoparticles comprising PYY, for example to treat obesity. Further combinations include NMU and exendin-4, and CCK and exendin-4.

The disclosure provides methods of treating the above mentioned diseases comprising the step of administering nanoparticles of the disclosure or a pharmaceutical composition of the disclosure, comprising a therapeutically effective amount of a biologically active polypeptide loaded therein, to a patient in need thereof.

The present disclosure also provides the use of nanoparticles of the disclosure as described herein or a pharmaceutical composition of the disclosure as described herein in the manufacture of a medicament for the treatment of the diseases and disorders listed herein.

The terms “individual”, “subject” and “patient” are used herein interchangeably. The subject is typically a human. The subject may also be a mammal, such as a mouse, rat or primate (e.g. a marmoset or monkey). The subject can be a non-human animal.

Treatment can be therapeutic, prophylactic or preventative. The subject will be one who is in need thereof. Those in need of treatment may include individuals already suffering from a particular medical disease in addition to those who may develop the disease in the future.

Within this specification the disclosure has been described, with reference to embodiments, in a way which enables a clear and concise specification to be written. It is intended and should be appreciated that embodiments may be variously combined or separated without parting from the disclosure.

### EXAMPLES

### Example 1

### Preparation of POCA Nanoparticles Loaded with Exendin-4

Summary of nanoparticle preparation process:
a) Dissolving octylcyanoacrylate (OCA) in an organic solvent to form a monomer solution;
b) Adding the monomer solution to an acidic aqueous solution containing a surfactant and a stabiliser under magnetic stirring to form an emulsion of organic droplets in an aqueous phase;
c) Adding an aqueous solution of a peptide to the emulsion;
d) Neutralising the emulsion on completion of the polymerisation reaction;
e) Allowing the organic phase to evaporate and thereby obtaining an aqueous suspension of poly(octylcyanoacrylate) (POCA) nanoparticles containing the peptide.

### Detailed Methodology

Loading the Poly(Octylcyanoacrylate) Nanoparticles with Exendin-4.

Poly(octylcyanoacrylate) nanoparticles containing exendin-4 were prepared as follows:

### Preparation of Organic and Aqueous Phases:

Aqueous phase: pH 2.0, 0.5% w/v dextran, 1.0% w/v PLURONIC™ F68 aqueous solution prepared by mixing:

8.5 mL H₂O (adjusted to pH 2.0 with 2M HCl)

1 mL PLURONIC™ F68 (10% stock solution)

500 μL dextran from L. Mesenteroides (10% w/v stock solution in water)

The solution was added to a 20 mL glass scintillation vial at room temperature and stirred using a magnetic stir bar (12 mm×8 mm, octagonal) at 800 rpm.
Organic phase: 100 mg/mL octylcyanoacrylate in ethyl acetate prepared by mixing:

100 μL octyl cyanoacrylate

1 mL ethyl acetate

The monomer may be dissolved in other organic solvents (for example dichloromethane, acetone and tetrahydrofuran) though it has been found that ethyl acetate gives the smallest and most reproducible particles size and as a class 3 solvent is less toxic than dichloromethane.

### Formation of the Emulsion:

The 1 mL of organic phase was added into the 10 mL of aqueous phase slowly, using a GILSON™ pipette, below the surface of the liquid.

Peptide solution:

10 mg/mL exendin-4 in water prepared by mixing:

1 mg exendin-4 peptide

100 μL H₂O

The 100 μL was added into the emulsion, quickly using a GILSON™ pipette, 60 minutes after the addition of the octylcyanoacrylate solution.

The peptide was added into the reaction 60 minutes after the monomer as this resulted in nanoparticles with the desired released characteristics. We have added peptide into the reaction at any point up to 60 minutes and successfully prepared nanoparticles, but have not investigated time points of greater than 60 minutes.

1 mg of exendin-4 was added per 100 mg of monomer (i.e. 1% w/w peptide with respect to monomer). Poly(octylcyanoacrylate) nanoparticles with peptide loadings of up to 10% w/w have been prepared successfully.

The solution was left to react uncovered for 6 hours.

### Neutralising the Solution:

After 6 hours of polymerisation of the octylcyanoacrylate to form poly(octylcyanoacrylate) nanoparticles the suspension was neutralised to pH 6 using sodium hydroxide (0.1M).

The organic phase was allowed to evaporate over night in a fume cupboard under constant stirring at 500 rpm.

### Purification of the Nanoparticles:

The nanoparticle suspension was filtered using vacuum filtration (Sintered filter—No3) to remove any large polymer aggregates. The filtrate was recovered and washed by centrifugation using VIVASPIN™ 20 concentrators (300K MWCO, 120 mins at 4000 g).

Nanoparticles prepared using the procedure described above were characterised using dynamic light scattering to measure the particle size and SDS PAGE to quantify the amount of peptide loaded. The hydrodynamic radius of the POCA nanoparticles ranged from 100 to 150 nm with an average of 126 nm. The polydispersity value for the nanoparticles ranged from 0.016 to 0.208; this is a measure of how broad the size range of particles in a sample is.

The amount of exendin-4 loaded into the particles ranged from 63 to 100 μg/mL (from a peptide input of 100 μg/mL). The method of quantification used with the SDS PAGE assay was variable, by up to approximately 10%, therefore some results obtained were over 100 μg/mL. For the purpose of dose calculations these batches were assumed to contain 100 μg/mL exendin-4.

### Characterisation of Nanoparticles:

DLS

The size of the nanoparticles was measured using dynamic light scattering (DLS) using a Brookhaven Instruments corporation particle size analyser (BIC 90plus) following the standard procedure provided by the manufacturer. The nanoparticle suspension was diluted 10× in filtered water sized using standard sizing parameters (temperature of 25° C., laser beam angle of 90°, laser wavelength of 658 nm). The particles were analysed by performing 10 sizing runs of 1 minute in duration each.

SDS PAGE

The amount of peptide loaded into the nanoparticles was quantified using SDS PAGE analysis. Briefly, the nanoparticle suspension was incubated at 37° C. for one hour with 0.1M sodium hydroxide to dissociate the polymer and release the peptide. A sample of the solution was then heated with loading buffer to 80° C. for 5 minutes. The sample was loaded onto a NUPAGE™ NOVEX™ 4-12% BisTris gel, with a prepared standard and molecular weight marker. The gel rig was set at 200V (400 mA) in 1×MES running buffer for 25 minutes and the protein bands visualised by staining with instant blue for 1 hour. Densitometry of the resulting bands was performed using an Odyssey LI-COR™ gel imaging systems to use the known peptide standards to quantify the peptide in the sample.

### Example 2

### Stability of POCA Nanoparticles

The ability of the POCA nanoparticles produced by the method of example 1 to protect an encapsulated peptide from degradation in the gastrointestinal tract was demonstrated by incubating the nanoparticles in simulated gastric and intestinal fluids formulated as follows, based on the TNO-TIM™ gut model system:

Simulated Gastric Fluid (SGF):

Gastric salt solution (10× concentrated) was prepared using 31 g (+/−0.5 g) sodium chloride, 11 g (+/−0.2 g) potassium chloride, 1.5 g (+/−0.03 g) calcium chloride dehydrate, made up to a total of 1020 g (+/−10 g) with purified water and ensuring the salts dissolved.

Gastric salt solution was then prepared using 51 g (+/−0.5 g) gastric salt solution (10× concentrated) and 3.58 g (+/−0.05 g) 1M sodium bicarbonate made up to 500 ml (+/−0.5 g) with purified water.

Gastric enzyme solution was freshly prepared using 150 g of gastric salt solution acidified to pH 5.0 with 1M HCl. 1125 units of lipase and 18000 units of pepsin were then dissolved in the gastric salt solution by gentle stirring and the solution stored on ice.

The SGF was then prepared by mixing 100 g of gastric salt solution with 170 g of tap water and 30 g of 0.1M sodium citrate buffer (pH7) and acidifying the solution to pH 2 with 1M HCl. 5 g (+/−0.2 g) of gastric enzyme solution were then mixed with 5 g (+/−0.2 g) of water and added to the mixture and the pH reconfirmed. The solution was then used immediately following preparation.

Simulated Intestinal Fluid (SIF):

Bile solution was prepared by gently adding, with continuous stirring, 2.0 g (+/−0.02 g) of bile powder into 250 g (+/−5 g) of purified water until a clear solution was obtained.

Pancreatin solution was prepared by adding 2.1 g (+/−0.2 g) of pancreatin powder to 150 g (+/−3 g) of purified water. A stirrer was used and care was taken to minimise foaming. Once a homogenous mixture was obtained, the solution was centrifuged at 3500 rpm for 20 minutes and the supernatant was then stored on ice.

Small intestine electrolyte solution (SIES) 25% (concentrated) was produced by adding purified water to 250 g (+/−5 g) sodium chloride, 30 g (+/−0.5 g) potassium chloride, and 15 g (+/−0.3 g) calcium chloride dehydrate to make a total of 2174 g. Once the salts had dissolved the pH was adjusted to pH7.0 (+/−0.5) with 1M sodium hydroxide.

SIES dilute was then prepared using 43.5 (+/−1 g) SIES concentrate added to purified water to a total weight of 1000 g.

Trypsin solution was prepared by dissolving 200 mg (+/−5 mg) of trypsin in 100 g (+/−2 g) of SIES dilute. This solution was then pipetted into 1.5 ml eppendorf tubes (1 ml per tube) and frozen at −20° C.

The SIF was then prepared by mixing 25 g (+/−0.3 g) of bile solution, 12.5 g (+/−0.3 g) pancreatin solution and 12.5 g (+/−0.5 g) of SIES dilute (ratio 2:1:1 bile/pancreatin/SIES dilute). 1 ml of trypsin solution was then added prior to the immediate use of the solution.

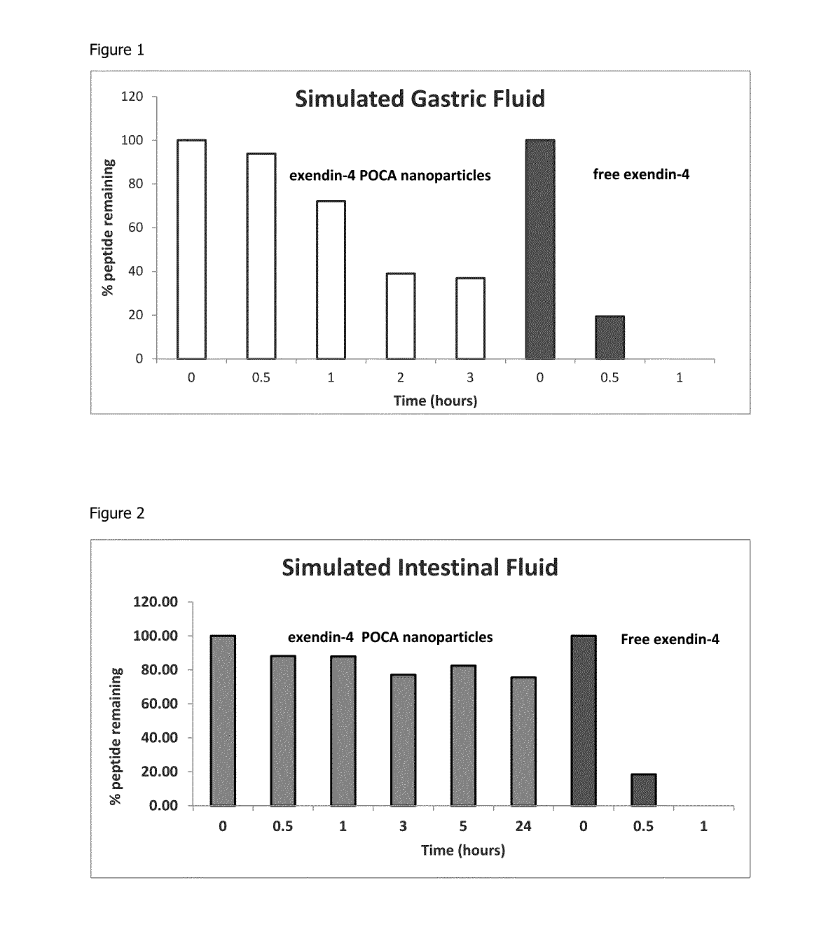

A portion of concentrated nanoparticles containing 150 μg/mL exendin-4 was incubated at 37° C. in either simulated gastric or intestinal fluids in comparison to a sample of the free peptide. Samples were removed at various time points and the peptide quantified using SDS PAGE. The amount of peptide remaining was compared to the initial amount of peptide as shown in FIGS. 1 and 2. In simulated gastric fluid (FIG. 1), 37% of the peptide remained intact after 3 hours. In the simulated intestinal fluid (FIG. 2) 76% of the peptide remained intact after 24 hours.

The data demonstrates that poly(octylcyanoacrylate) nanoparticles of less than 200 nm can be prepared reproducibly. Particles of this size have the potential to be absorbed through the GI tract into systemic circulation therefore these particles could be used for the oral delivery of peptides.

The particles can consistently be loaded with at least 63 μg/mL of exendin-4 and the stability studies have shown that the polymer nanoparticle can protect the peptide from enzymatic degradation in gastric and intestinal fluids to allow oral delivery.

### Example 3

### In Vivo Analysis of POCA Nanoparticles Loaded with Exendin-4 (i.v. Administration): Reduction in Blood Glucose Levels

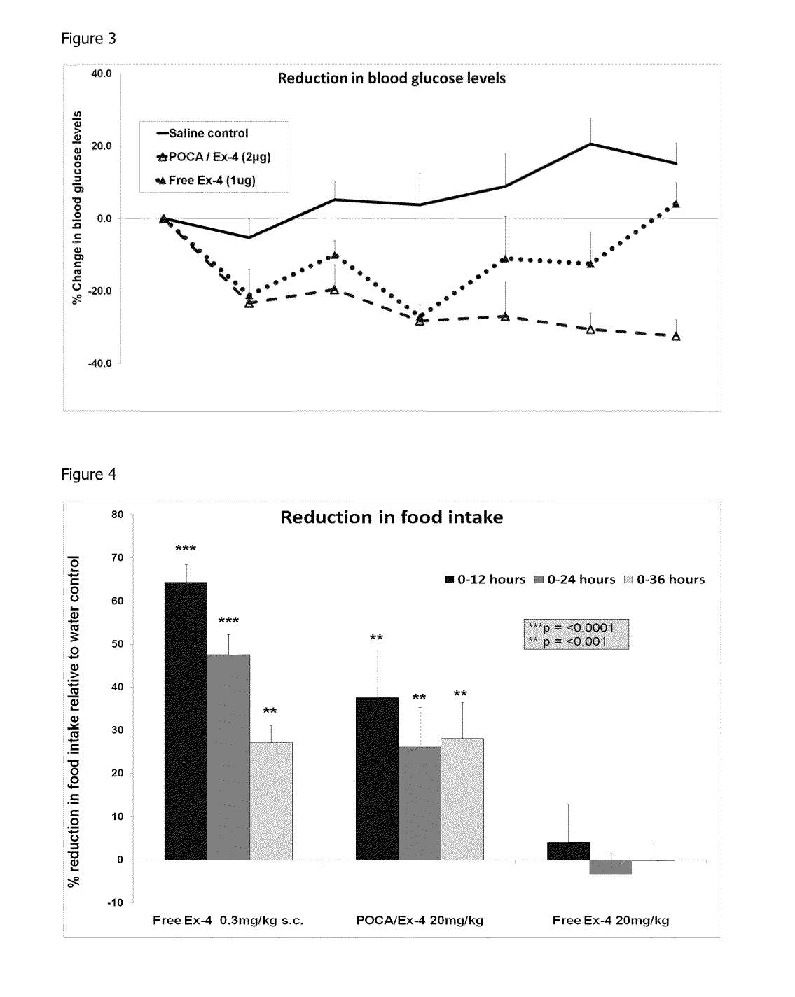

By utilising the blood glucose modulating property of exendin-4, an in vivo study was carried out to demonstrate the release of functional exendin-4 from the POCA nanoparticles, produced by the method of example 1, upon entry into the bloodstream. C57BL/6 mice were given a single i.v. dose of exendin-4 loaded POCA nanoparticles (equivalent to 2 μg peptide/100 μl), non encapsulated (free) exendin-4 peptide (equivalent to 1 μg/100 μl) or saline as a control. In order to monitor blood glucose levels, 2-3 μl blood samples were taken from the peripheral tail vein of each animal prior to dosing and again at 0.5, 1, 2, 3, 4 and 8 hours post dose. Each sample was analysed using a hand held glucose monitor (BAYER ASCENSIA BREEZE 2™). Values were expressed as a percentage change in glucose levels from baseline and the results are shown in FIG. 3.

Significant reductions in blood glucose levels from baseline were observed in animals receiving both the encapsulated and non encapsulated forms of exendin-4 (˜30% change by 2 hours post dose). This reduction in blood glucose levels was maintained in the nanoparticle treated group for up to 8 hours post dose, whereas in the group receiving the non encapsulated peptide, glucose levels had returned to baseline by this time point. This is suggestive of a longer duration of effect from the peptide delivered via the nanoparticles, indicative of sustained release.

### Example 4

### In Vivo Analysis of POCA Nanoparticles Loaded with Exendin-4 (Oral Administration): Reduction in Food Intake in a Fed Mouse Model

To demonstrate a reduction in food intake due to appetite suppression, studies were undertaken in a murine model of food intake inhibition in both fed and fasted animals. In such studies animals were offered food immediately following the oral administration (gavage) of exendin-4 loaded POCA nanoparticles produced by the method of example 1. Data demonstrated here relate only to the fed mouse model, but are representative of the observations made in the studies using fasted animals.

The body weights of singly housed 8-10 week old male C57/BL6 mice were measured and ranked. Animals were assigned to treatment groups such that an even distribution of weights across all groups was achieved. Immediately prior to commencement of the evening light cycle, animals were given a single dose of one of the following treatments; water (p.o.), non-encapsulated (free) exendin-4 (20 mg/kg p.o.), exendin-4 in POCA nanoparticles (20 mg/kg p.o.) or non encapsulated exendin-4 (0.3 mg/kg s.c.). Directly following treatment, all food was removed and replaced with a measured amount of standard diet (approximately 100 g). Following the 12 hour dark cycle, the weight of remaining diet was recorded and returned to the animals. This process was repeated after 24 and 36 hours post dose. The results are shown in FIG. 4 and are expressed as a percentage reduction in food intake relative to the water control group.

Results clearly demonstrate the benefit of delivering peptide by the oral route in this encapsulated form. Compared to an equivalent dose of non-encapsulated peptide, there is a significant reduction in food intake at all the time points considered (p=<0.001). Although this level of food intake inhibition does not appear to be as significant as for the subcutaneously administered peptide, the profile of response over the 36 hour period monitored is suggestive of sustained release of peptide from the nanoparticles.

### Example 5

### In Vivo Analysis of POCA Nanoparticles Loaded with Exendin-4 (Oral Administration): Reduction in Food Intake and Body Weight in a Fed Mouse Model—Dose Range Study

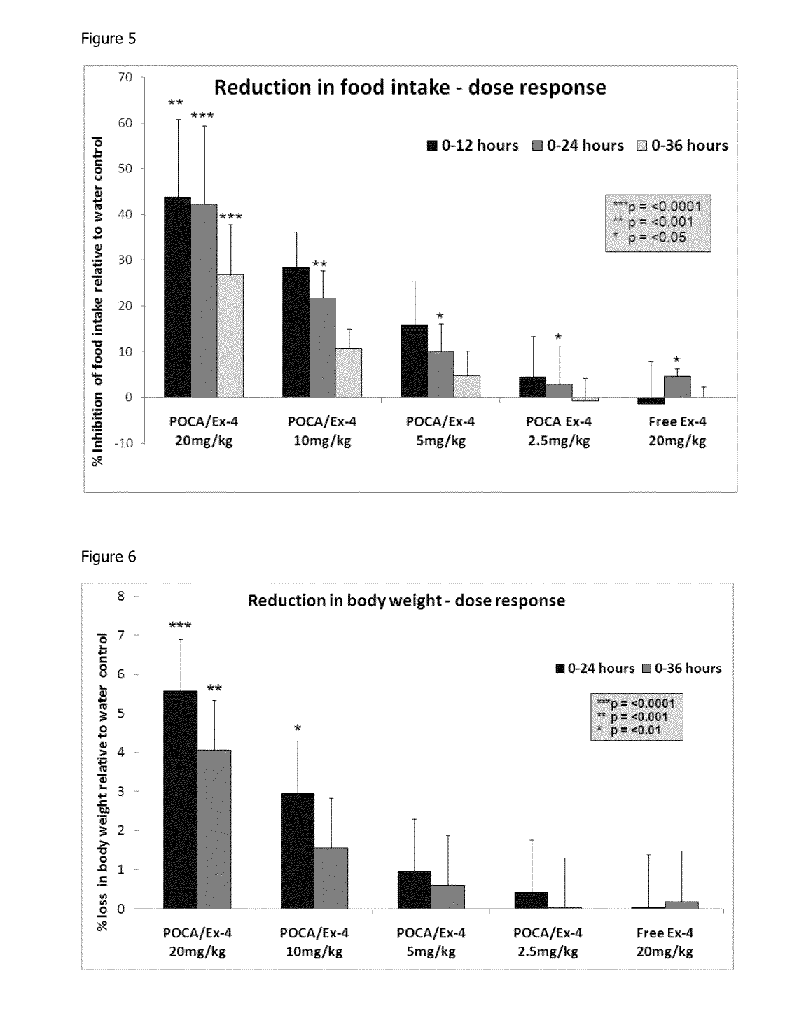

The fed mouse model as described in Example 4 was used. However, in this study animals were given a single oral dose (gavage) of exendin-4 in POCA nanoparticles of 20, 10, 5, 2.5 mg/kg or a single oral dose (gavage) of non encapsulated (free) exendin-4 of 20 mg/kg. Animal body weights were also recorded at 24 and 36 hours post dose. The food intake results are shown in FIG. 5 and the body weight results are shown in FIG. 6.

As demonstrated in Example 4 above and again in the present example, encapsulation of exendin-4 in POCA nanoparticles clearly offers protection over non encapsulated material when given by the oral route. Here (FIG. 5), there is again a clearly significant reduction in food intake (p=<0.001 at 0-12 hrs and p=<0.0001 at 12-24 and 24-36 hours), when given at the higher dose of 20 mg/kg.

The results in FIG. 6 also show that weight loss following a single dose of exendin-4 loaded POCA nanoparticles was highly significant after 24 hours post dose with an average weight loss of 5.5% (p=<0.0001) and after 36 hours post dose, with an average weight loss of 4% (p=<0.001) when given at 20 mg/kg. Significant weight loss was also observed after 24 hours (3%, p=<0.01) when the peptide was dosed at 10 mg/kg in POCA nanoparticles.

### Summary of Examples 1-5

The in vivo studies reported here have demonstrated the release of functional peptide, in this case exendin-4, from the POCA nanoparticles upon entry in to the bloodstream. Furthermore, oral administration of theses nanoparticles has resulted in significant reductions in food consumption and body weight in both fed and fasted murine models of food intake inhibition, compared to free exendin-4. Accordingly, these examples support the use of orally administered peptide loaded POCA nanoparticles in therapy, in particular exendin-4 loaded POCA nanoparticles for the treatment of metabolic disorders, such as obesity.

SEQUENCE CONCORDANCE

| SEQ ID NO | Identifier |
|---|---|
| 1 | Exendin-4 (1-39) |

1. A nanoparticle comprising a biologically active polypeptide wherein the nanoparticle comprises poly(octylcyanoacrylate).

2. A nanoparticle as claimed in claim 1, which is to be administered orally.

3. A nanoparticle as claimed in claim 1, wherein the biologically active polypeptide comprises 70 or fewer amino acid residues.

4. A nanoparticle as claimed in any one of claim 1, wherein the biologically active polypeptide comprises a metabolic peptide.

5. A nanoparticle as claimed in claim 4, wherein the metabolic peptide is an insulinotropic peptide or an incretin.

6. A nanoparticle as claimed in claim 4, wherein the metabolic peptide is selected from the group consisting of a GLP-1 agonist peptide, PYY, NMU, and CCK.

7. A nanoparticle as claimed in any one of claim 4, wherein the metabolic peptide is exendin-4.

8. A nanoparticle as claimed in claim 7, wherein the nanoparticle has a hydrodynamic diameter of 300 nm or less.

9. A population of nanoparticles comprising nanoparticles as claimed in claim 8, wherein at least 90% of nanoparticles by number have a hydrodynamic diameter within 10 nm to 200 nm as measured by dynamic light scattering techniques.

10. A method of producing nanoparticles comprising the steps of:
a) dissolving octylcyanoacrylate in an organic solvent to form a monomer solution;
b) adding the monomer solution from step (a) to an acidic aqueous solution to form an emulsion of organic droplets in an aqueous phase; and simultaneously or sequentially
c) adding an aqueous solution of a biologically active polypeptide to the emulsion from step (b) and allowing polymerisation of the monomer;
d) allowing the organic phase to evaporate, thereby obtaining an aqueous suspension of poly(octylacrylate) nanoparticles containing the polypeptide.

11. A method as claimed in claim 10, wherein the organic solvent is selected from the group consisting of: ethylacetate, dichloromethane, and chloroform.

12. A method as claimed in claim 11, wherein the aqueous solution of step (b) comprises a surfactant.

13. A method as claimed in claim 12, wherein the aqueous solution of step
(b) comprises a stabiliser.

14. A method as claimed in claim 13, wherein the surfactant is any one selected from the group consisting of: a poloxomer, a polysorbate surfactant, a macrogol ether surfactant, polyvinyl alcohol, and polyvinylpyrrolidone.

15. A method as claimed in claim 13, wherein the stabiliser is any one selected from the group consisting of: dextran, chitosan, fucoidan, pectin, glycogen, amylase, and amylopectin.

16. A method as claimed in claim 15, wherein a step to neutralise the emulsion is included between steps (c) and (d).

17. A method as claimed in claim 16, wherein the emulsion is neutralised using sodium hydroxide.

18. A method as claimed in claim 17, wherein the ratio of polypeptide to monomer is 1-10% w/w.

19. A pharmaceutical composition comprising nanoparticles as claimed in claim 9.

20. A pharmaceutical composition as claimed in claim 19, further comprising oligofructose.

21. A use of a nanoparticle as claimed in claim 8, for treating any one or more of the following metabolic disorders:
a disorder associated with elevated glucose levels, diabetes (type 1 or 2 or gestational), metabolic syndrome, hyperglycemia, impaired glucose tolerance, beta cell deficiency and a disease characterised by or associated with overeating, such as obesity, wherein the biologically active polypeptide is a metabolic peptide.

22. A use of a nanoparticle, a population of nanoparticles, or a pharmaceutical composition as claimed in claim 21 for treating obesity, wherein the metabolic peptide is exendin-4.

23. A method of treating a subject having any one or more of the following metabolic disorders: a disorder associated with elevated glucose levels, diabetes (type 1 or 2 or gestational), metabolic syndrome, hyperglycemia, impaired glucose tolerance, beta cell deficiency and a disease characterised by or associated with overeating, such as obesity, by administering a therapeutically effective amount of a population of nanoparticles as claimed in claim 9 to the subject.

24. A pharmaceutical composition comprising nanoparticles produced by the method of claim 18.


SEQUENCE LISTING (1 sequence)

SEQ ID NO: 1 - protein, 39 aa, Artificial Sequence
  Exendin-4 (1-39)
HGEGTFTSDLSKQMEEEAVRLFIEWLKNGGPSSGAPPPS